(19) United States
(12) Patent Application Publication
(10) Pub. No.: US 2013/0110086 A1
(43) Pub. Date: May 2, 2013

(54) BULGED CATHETER TIP

(71) Applicants: Neha S. Bhagchandani, Bloomington, IN (US); Michael R. Kurrus, Ellettsville, IN (US); Ryan Nowicki, Indianapolis, IN (US)

(72) Inventors: Neha S. Bhagchandani, Bloomington, IN (US); Michael R. Kurrus, Ellettsville, IN (US); Ryan Nowicki, Indianapolis, IN (US)

(21) Appl. No.: 13/616,737

(22) Filed: Sep. 14, 2012

Related U.S. Application Data
(60) Provisional application No. 61/553,633, filed on Oct. 31, 2011.

(51) Int. Cl. A61M 25/00; B29C 65/00

(52) U.S. Cl. 604/531; 604/523; 156/296

(57) ABSTRACT

Among other things, a catheter assembly including a main catheter portion and a deformable tip portion is disclosed. Embodiments of the catheter have a lumen extending between distal and proximal ends. The deformable portion is configured to transform from a compact shape to an expanded shape when it is inserted into a patient. The cross-sectional sizes and shapes of the deformable portion and catheter are similar or about the same; therefore, the compact shape mimics a traditional distal tip of a catheter during insertion into a patient. The physician can use a standard catheter insertion technique. As the deformable portion warms to the patient's body temperature, the deformable portion bulges to the expanded shape. The expanded shape helps attain laminar flow and increases the flow rate through the catheter. To activate the deformable portion to return to the compact shape, cold liquid is passed through the deformable portion.

### REFERENCE TO RELATED APPLICATION

This application claims the benefit of U.S. Provisional Application No. 61/553,633, filed Oct. 31, 2011, which is hereby incorporated by reference.

### BACKGROUND

The present disclosure relates to a catheter assembly and method of manufacturing the catheter assembly. More particularly, the present disclosure relates to a catheter assembly having a deformable portion at the distal end of the catheter that is moveable from a compact shape for implantation into a patient to an expanded shape after the deformable portion is positioned within the vasculature of a patient. The deformable portion is moveable from the expanded shape to the compact state upon temperature activation for removal from the patient.

Central venous catheters (“CVC”) include catheters designed to enter and utilize the central veins (e.g., subclavian and superior vena cava) or right cardiac chamber(s) for the delivery and/or withdrawal of blood, blood products, nutritional products, therapeutic agents, drugs, hemodialysis, and other therapeutic techniques that may be necessary for a patient. Some examples of CVCs include standard central venous catheters for intravenous access, dialysis catheters, percutaneously-introduced central catheters (“PICC” lines), and right heart catheters, to name a few.

One example includes a dialysis catheter that provides for the removal or aspiration of blood that is cleansed by a dialysis machine and for the return of the cleansed blood to the patient. One type of dialysis catheter includes a single-bodied catheter with two separate lumens wherein one lumen is used to remove the blood and the second lumen is used to return the cleansed blood to the patient. The lumens are often referred to as an arterial lumen and a venous lumen. Another type of dialysis catheter includes a single catheter with a single lumen. In this arrangement, a dialysis machine receives a quantity of untreated blood from the body and then returns treated blood in alternating cycles through the single lumen.

One problem associated with dialysis catheters is that as the dialysis machine aspirates blood through the arterial lumen (or single lumen), the catheter tip and its opening tends to move or get “sucked up” against the vessel wall. The displacement of the catheter tip's opening toward or against the vein wall reduces or minimizes the amount of the opening available for flow, resulting in inferior flow rates into or out of the catheter and limiting or interfering with the dialysis process. The reduced opening and/or inferior blood flow rates through the lumen can mean that laminar flow through the catheter is not achieved. Non-laminar blood flow can present an increased risk of thrombosis or blood clotting or thickening. As can be appreciated, thrombosis can at least interfere with normal blood flow and can be a source of problematic or potentially deadly emboli. Additionally, a displaced catheter tip resting against the vessel wall and aspirating or returning fluid through an opening can cause trauma to the vessel tissue, at least through irritation from corners or edges of tubing.

Thus, there is a need for improvement in this field.

### SUMMARY

This Summary is provided merely to introduce certain concepts and not to identify any key or essential features of the claimed subject matter.

In certain of its aspects, the present disclosure features embodiments of a catheter assembly including a catheter and a deformable portion. The catheter has a distal end opposite a proximal end and a lumen extending between the distal end and the proximal end. The deformable portion is attached to the distal end of the catheter. The deformable portion has a compact shape at a first temperature and an expanded shape at a second temperature, wherein the second temperature is greater than the first temperature. The deformable portion in particular embodiments has a proximal end, a distal end, a mid-section between them and a lumen, with the proximal end of the deformable portion attached to the distal end of the catheter so that the catheter lumen and the lumen of the deformable portion communicate with each other at a common diameter. The distal end of the deformable portion has an opening to its lumen. In the expanded shape the mid-section may have a diameter larger than the distal end of the deformable portion and the proximal end of the deformable portion, so that when the deformable portion is within a body, the expanded mid-section has a convex external surface that maintains at least part of the opening away from a tissue surface.

In certain embodiments, the second temperature is about 37 degrees Celsius. In one form, the expanded shape of the deformable portion is a pear shape. In one embodiment, the deformable portion is made of a shape memory polymer material. In one embodiment, the mid-section is configured to expand more than the proximal end of the deformable portion. In another embodiment, the distal end of the deformable portion is configured to expand more than the proximal end of the deformable portion or tube.

In other of its aspects, the present disclosure features a catheter assembly including a catheter and a deformable portion. The catheter includes a distal end opposite a proximal end and a lumen extending between the distal end and the proximal end. The deformable portion is attached to the distal end of the catheter. Further, the deformable portion is configured to transform from a compact shape to an expanded shape when the deformable portion is heated to a temperature of about 37 degrees Celsius. In one form, the compact shape of the deformable portion and the distal end of the catheter each have a cross-sectional shape and cross-sectional size, wherein the shapes are about the same and the sizes are about the same.

In particular, such a deformable portion can have a proximal end, a distal end, a mid-section between them and a lumen. The proximal end of the deformable portion is attached to the distal end of the catheter so that the lumens of the catheter and deformable portion communicate with each other at a common diameter. The distal end of the deformable portion has an opening to its lumen. In the expanded shape the mid-section has a diameter larger than the distal end of the deformable portion and the proximal end of the deformable portion, so that when the deformable portion is within a body, the expanded mid-section has a convex external surface that maintains at least part of the opening away from a tissue surface. From the expanded state, the deformable portion is configured to transform to a compact shape (e.g. substantially cylindrical) when the deformable portion is cooled to a temperature less than about 37 degrees Celsius.

In certain of its aspects, the present disclosure features embodiments of methods for making a catheter assembly. In particular embodiments, such methods include attaching a deformable portion made of a shape memory polymer material to a distal end of a catheter, the deformable portion being in a compact shape. The deformable portion is heated to about 37 degrees Celsius. Next, the deformable portion is transformed from a compact shape to an expanded shape. Finally, the deformable portion is cooled to less than 37 degrees Celsius wherein the deformable portion returns to the compact shape. In one embodiment, a proximal end of the deformable portion is attached to the distal end of the catheter. In another embodiment, the heating step includes curing the deformable portion. Methods of using a catheter assembly are also disclosed.

Further forms, objects, features, aspects, benefits, advantages, and embodiments of the present disclosure will become apparent from a detailed description and drawings provided herewith.

### BRIEF DESCRIPTION OF THE DRAWINGS

FIG. 1 is a top view of one embodiment of a central venous catheter.

FIG. 2 is a cross-sectional view of the FIG. 1 embodiment taken along the lines 2-2 in FIG. 1.

FIG. 3 is a partial view of the FIG. 1 embodiment depicting a deformable portion or tube in a compact original shape.

FIG. 4 is a partial view of the FIG. 3 embodiment depicting the deformable portion or tube in an expanded shape.

### DESCRIPTION OF THE SELECTED EMBODIMENTS

For the purpose of promoting an understanding of the principles of the disclosure, reference will now be made to the embodiments illustrated in the drawings and specific language will be used to describe the same. It will nevertheless be understood that no limitation of the scope of the claims is thereby intended. Any alterations and further modifications in the described embodiments, and any further applications of the principles of the disclosure as described herein are contemplated as would normally occur to one skilled in the art to which the disclosure relates. One embodiment is shown in great detail, although it will be apparent to those skilled in the relevant art that some features that are not relevant to the present disclosure may not be shown for the sake of clarity.

As noted above, in certain aspects, the present disclosure provides unique products and methods for positioning a catheter, such as a CVC, dialysis or other type of catheter assembly, within a patient (e.g. the vasculature of a patient). CVCs are used for numerous reasons involving continuous out- and in-flow in a patient's body such as feeding, drug delivery, and hemodialysis, to name a few examples. Embodiments of a catheter assembly can include a catheter having a distal end and an opposite proximal end with a single lumen extending therebetween. Other embodiments can include dual or multiple lumens. A deformable portion or tube is formed, attached, connected, or bonded to the distal end of the catheter. Embodiments of the deformable portion or tube are made of a shape memory polymer material such that the deformable portion or tube is moveable between a compact original shape and an expanded shape upon temperature activation as described in more detail below.

Shape memory polymer materials are polymeric materials that have the ability to return from a deformed state or temporary shape to their original or permanent shape when induced by an external stimulus. Once the permanent shape has been manufactured by conventional methods, the material is changed into another, temporary form by processing through heating, deformation, and finally, cooling. The polymer maintains this temporary shape until the shape change into the permanent form is activated by the external stimulus.

The basic thermomechanical response of shape memory polymers is defined by four critical temperatures. The glass transition temperature, T_{g}, is typically represented by a transition in modulus-temperature space and can be used as a reference point to normalize temperature. Shape memory polymers offer the ability to vary T_{g} over a temperature range of several hundred degrees by control of chemistry or structure. The deformation temperature, T_{d}, is the temperature at which the polymer is deformed into its temporary shape. The storage temperature, Tₛ, represents the temperature in which no shape recovery occurs and is equal to or below T_{d}. At the recovery temperature, Tᵣ, the shape memory effect is activated, which causes the material to recover its original shape, and is typically in the vicinity of T_{g}. Recovery can be accomplished isothermally by heating to a fixed Tᵣ and then holding, or by continued heating up to and past Tᵣ.

The microscopic mechanism responsible for shape memory in polymers depends on both chemistry and structure of the polymers. If the polymer is deformed into its temporary shape at a temperature below T_{g}, or at a temperature where some of the hard polymer regions are below T_{g}, then internal energy restoring forces will also contribute to shape recovery. In either case, to achieve shape memory properties, the polymer must have some degree of chemical crosslinking to form a “memorable” network or must contain a finite fraction of hard regions serving as physical crosslinks.

A polymer is a shape memory polymer if the original shape of the polymer can be recovered by application of a stimulus, e.g., by heating it above a shape recovery temperature, or deformation temperature (T_{d}), even if the original molded shape of the polymer is destroyed mechanically at a lower temperature than T_{d}. The original shape is set by processing and the temporary shape is set by thermo-mechanical deformation. A shape memory polymer has the ability to recover from large deformation upon heating. The present disclosure includes a deformable portion or tube made from shape memory polymer materials which can be inserted into the vasculature of a patient or other body cavity in a compact shape and then expand or bulge to the expanded shape by increasing the temperature of the deformable portion or tube to the patient's body temperature.

Some examples of shape memory polymer materials include, but are not limited to, polyurethane, polyurethanes with ionic or mesogenic components, block copolymers consisting of polyethylene terephthalate (PET) and polyethylene oxide (PEO), block copolymers containing polystyrene and poly(1,4-butadiene), and an ABA triblock copolymer made from poly(2-methyl-2-oxazoline) and poly(tetrahydrofuran), and chemically crosslinked shape memory polymer materials. Other non-limiting examples of shape memory polymer materials and techniques for manufacturing the deformable portion or tube are described in U.S. Publication No. 2009/0248141.

In the illustrated embodiment, the deformable portion or tube includes a compact original shape having a cylindrical shape wherein the inner diameter of the deformable portion or tube is about the same size as the diameter of the lumen in the distal end of the catheter. Moreover, the wall thickness of the deformable portion or tube is about the same as the wall thickness of the distal end of the catheter. In this embodiment, the cross-sectional shape and size of the deformable portion or tube and the distal end of the catheter are similar or identical to each other. In other embodiments, the deformable portion may have a different shape and/or size than the distal end of the catheter. A few examples of the compact original shape for the deformable portion or tube include cylindrical (e.g. the illustrated embodiment), tapered, conical, or frustoconical. Beneficially, the deformable portion or tube functions as an extension of the catheter when the deformable portion or tube is attached to the catheter. As such, the initial compact shape of the deformable portion or tube does not interfere with positioning of the catheter assembly within the vasculature or other locations within a patient. Moreover, the placement of catheter with a deformable portion or tube in the patient can be accomplished with many well-known surgical techniques. After placement of the deformable portion or tube within the vasculature of the patient, the body heat from the patient warms the deformable portion to Tᵣ or about 98.6 degrees Fahrenheit or 37 degrees Celsius.

As the deformable portion reaches a temperature, Tᵣ, of about 98.6 degrees Fahrenheit or 37 degrees Celsius, the deformable portion will bulge or expand to the expanded shape. In one embodiment, the deformable portion will bulge or expand to the expanded shape in approximately 30 seconds to about 1 minute after placement in the vasculature of the patient. Moreover, the time required to enable the deformable portion to bulge or expand to the expanded shape can be increased if the deformable portion is cooled to a temperature below room temperature prior to placement in the vasculature of the patient. Often typical room temperatures range from about 19 to 22 degrees Celsius; therefore, the deformable portion is cooled to a temperature less than room temperature. The deformable portion or tube can be configured to expand longitudinally and/or laterally with respect to a longitudinal axis of the catheter. The expansion of the deformable portion or tube can be linear or nonlinear. The expanded shape of the deformable portion can be any desired shape, and particular examples of desirable shapes include round, bell, oval, or another outwardly curved shape. In the illustrated embodiment, the distal end of the deformable portion expands relative to the main catheter portion, so that the deformable portion's distal opening is greater in width or diameter than that of the lumen of the main catheter portion. Such a larger opening of the distal end of the deformable portion as compared to the diameter of the lumen of the catheter assists in achieving laminar flow of the fluid (e.g. blood) into or out of it. As can be appreciated, laminar blood flow through the catheter opening reduces the risk of thrombosis. Frequently during aspiration a standard or traditional catheter tip gets sucked up against the vessel wall and thereby damages the vessel wall. In the present disclosure, if there is contact between the vessel wall and the expanded shape of the deformable portion, the round shape of the deformable portion will not damage the vessel wall upon contact. Moreover, the warm deformable portion in an expanded shape is soft compared to a straight, standard catheter tip, which is much stiffer or harder. The soft deformable portion in an expanded shape will not damage the vessel wall upon contact. Comparably, straight, standard catheter tips that contact the vessel wall may cause vessel trauma as edges or corners rub or press against the vessel. To remove the catheter and deformable portion from the patient, cold fluid is passed through the catheter and the deformable portion to cool the temperature of the deformable portion to about or below Tₛ and cause the deformable portion to shrink or return to the compact original shape. In one embodiment, the cold fluid that is passed through the catheter assembly has a temperature of about 20 to 30 degrees Celsius. Additionally, the amount of cold fluid will vary as required for each patient's medical condition such that the patient is tolerant of the cold fluid and the cold fluid is not detrimental to the patient. In yet another embodiment, the amount of cold fluid can range from about 125 milliliters to about 200 milliliters. The time required to pass this amount of cold fluid through the catheter assembly will vary as limited by the medical condition of the patient and the flow rate of the catheter assembly. For example, the time required to pass an amount of cold fluid of about 125 milliliters to about 200 milliliters ranges from approximately 8 seconds to about 5 minutes.

Catheter assembly 100 is provided for illustrative purposes, and those of ordinary skill in the art appreciate that alternate embodiments of catheter assembly 100, including embodiments with additional lumens and the like, are within the scope of the present disclosure. As indicated in the drawings and discussion below, an example of catheter assembly 100 having only one lumen is provided. In other examples, catheter assembly 100 can include two or more lumens. In such examples, there may be separate deformable portions for each lumen portion, one deformable portion having multiple lumens. It is contemplated that a portion of a catheter tip may be non-deformable along with a deformable portion as discussed herein, although that formulation may not provide the full benefit of other embodiments. In the following discussion, the terms “proximal” and “distal” will be used to describe the axial ends of the apparatus as well as the axial ends of various component features. The term “proximal end” refers to the end of the catheter assembly 100 that is closest to the operator during use of the assembly. The term “distal end” refers to the end of the catheter assembly 100 that is inserted into the patient or that is closest to the patient. The following discussion will also focus on vascular uses of catheter assembly 100, although it will be understood that medical uses in other parts of the body are possible.

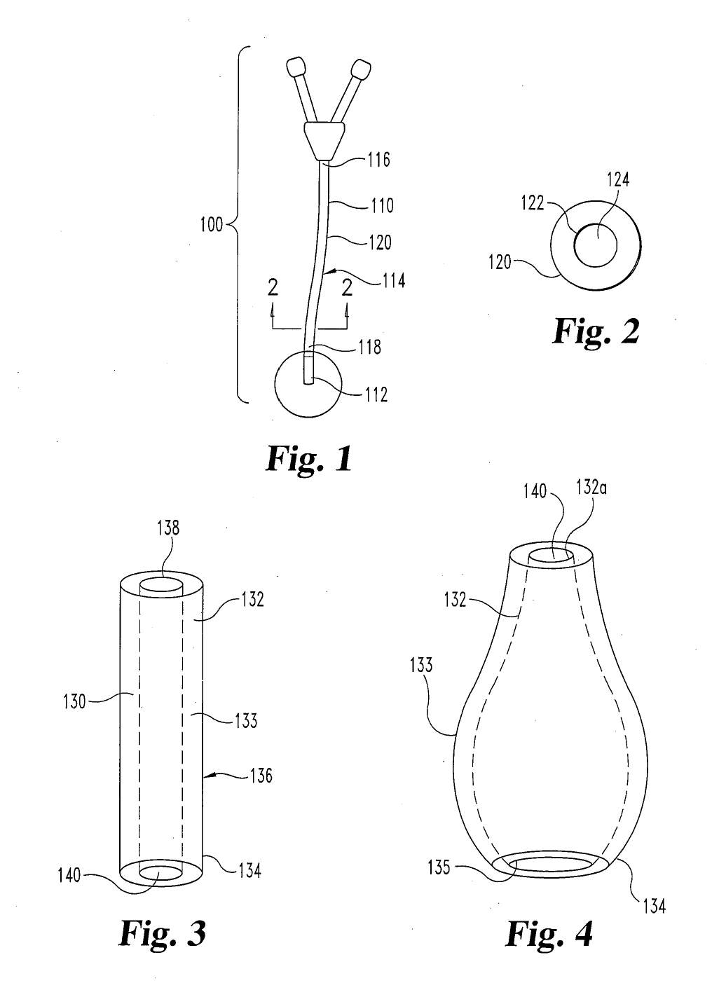

In an embodiment illustrated in FIG. 1, the catheter assembly 100 includes a catheter (or main catheter portion) 110 and a deformable portion or tube 112. As illustrated in FIG. 1, deformable portion or tube 112 has an original compact shape or configuration for placement within the vasculature of a patient. The deformable portion or tube 112 is configured to expand, as exemplified in FIG. 4, after the deformable portion or tube 112 has been placed within the patient. One benefit of the expanded shape of deformable portion or tube 112 is the expanded shape illustrated in FIG. 4 enables the deformable portion or tube 112 to be centered in the vein or vessel. Further, the deformable portion or tube 112 is configured to contract or return to or toward the original compact shape (e.g. as illustrated in FIG. 1) upon temperature activation when it is desired to remove the catheter assembly 100 from the patient. It should be appreciated that other shapes for deformable portion or tube 112 can be contemplated than those illustrated in FIGS. 1 and 4.

Catheter 110 includes an elongate flexible body 114 having a proximal end 116 and an opposite distal end 118. As illustrated in FIG. 2, the catheter 110 defines an outer surface 120 and an inner surface 122 surrounding a lumen 124. In one form, the thickness of the catheter 110 from the inner surface 122 to the outer surface 120 is about 0.010 inch. Lumen 124 has a diameter (i.e., the internal diameter of the inner surface 122) that is substantially uniform through the length. In some embodiments, useful diameters of lumen 124 range from about 0.05 inch to about 0.2 inch. Of course, there may be applications that require larger or smaller dimensions for catheter 110. The catheter 110 can be curved or straight as may be desired or necessary for a particular medical procedure. It will be understood that other embodiments may have dual lumens (side-by-side, one within another or coaxial) or multiple lumens.

Catheter 110 can be made from any suitable biocompatible material, including silicone, polyurethane, polyurethane-polycarbonate copolymer, or any other plastic or polymer material. Particular embodiments of catheter 110 include an antibacterial coating on part or all of surfaces 120 and/or 122. Catheter 110 can also be treated with an anti-infection agent, such as methylene blue, for example. Additionally, outer surface 120 and/or inner surface 122 of catheter 110 can be coated with a biocompatible substance, particularly an anticoagulating substance, such as heparin, urokinase, or other therapeutic substances. Catheter 110 can be of any suitable size for placement in a vessel structure, and particular sizes for catheter 110 range from 3 to 16 French. Other sizes could also be contemplated. The outer surface 120 of the catheter 110 is cylindrical in the illustrated embodiment, and in other embodiments can be D-shaped, double D-shaped, or split, for example.

In some embodiments, the catheter 110 is made of or includes a biocompatible radiopaque material so as to give the physician the option to visualize catheter 110 by fluoroscopy or X-rays. For example, catheter 110 can be made of any biocompatible material in which barium sulfate or another radiopaque material is mixed or suspended. As another example, distal end 118 of catheter 110 may be configured to include a guidance element for visualizing, guiding, and/or positioning the rotational orientation of catheter 110 within the vasculature of a patient. Such guidance elements include one or more markers, sensors, and/or emitters. For instance, distal end 118 and/or other part(s) of catheter 110 may include one or more radiopaque or echogenic markers (e.g., bead(s) or surface(s) of biocompatible metal) to permit visualization or other location of such part(s), in particular their position and/or orientation within a patient's body.

As illustrated in FIG. 3, deformable portion or tube 112 has an original compact shape. Further, deformable portion or tube 112 includes a flexible body 130 having a proximal end 132 with an opening 132a that communicates with lumen 124, a mid-section 133, and an opposite distal end 134 with an opening 135. Deformable portion or tube 112 defines an outer surface 136 and an inner surface 138 surrounding a passageway 140. Passageway 140 has a diameter (i.e., the internal diameter of inner surface 138). In one form, the thickness of deformable portion or tube 112 from inner surface 138 to outer surface 136 is about the same as the thickness of body 114 from inner surface 122 to outer surface 120. Further in this embodiment, the diameter of opening 132a and passageway 140, as well as opening 135 in a compact or unexpanded condition, is about the same as the diameter of lumen 124. In one embodiment, the thickness of deformable portion or tube 112 is about 0.010 inch. In other embodiments, the thickness of deformable portion or tube 112 from the inner surface 138 to the outer surface 136 can be greater or less than the thickness of the catheter body 114.

Deformable portion or tube 112 is made of a shape memory polymer material wherein the material properties of the shape memory polymer material can be adjusted or set to achieve a desired shape or configuration. The shape memory polymer material enables deformable portion or tube 112 to retain two shapes and transition between those shapes when a change in temperature occurs in deformable portion or tube 112. As such, deformable portion or tube 112 is configured to change shape from an original compact shape to an expanded shape when the temperature of deformable portion or tube 112 reaches Tᵣ or about 37 degrees Celsius. In one embodiment, at a temperature less than 37 degrees Celsius or Tₛ, deformable portion or tube 112 is in or reconfigures toward the original compact shape, one form of which is a substantially cylindrical or tube shape as illustrated in FIG. 3. Other forms or configurations of an original compact shape for deformable portion 112 are D-shaped, double D-shaped, or other shape(s) that may be desired or required to attach deformable portion 112 to distal end 118 of catheter body 114. When deformable portion or tube 112 is in the original compact shape, the cross-sectional shapes and sizes of proximal end 132 of deformable portion or tube 112 and distal end 118 of catheter 110 are similar or about the same. Further, the circumference of outer surface 136 of deformable portion 112 is sized similarly to the circumference of outer surface 120 of catheter 110. The cross-sections of proximal end 132 of deformable portion 112 in the original compact shape and of distal end 118 of catheter 110 are about the same size in the illustrated embodiment, and so there is little or no change in cross-sectional size or shape between deformable portion 112 and catheter 110. Therefore, deformable portion or tube 112 mimics a traditional catheter tip during placement in the vasculature of a patient and the physician can use standard catheter insertion techniques to place catheter assembly 100 in the vasculature of the patient.

However, after the deformable portion or tube 112 is positioned within the vasculature of the patient, the patient's body heat warms the deformable portion or tube 112 to about 98.6 degrees Fahrenheit (about 37 degrees Celsius). As mentioned previously, in one embodiment, the time required for the deformable portion to bulge or expand to the expanded shape is approximately 30 seconds to about 1 minute after placement in the vasculature of the patient. Moreover, the time required for the deformable portion to bulge or expand to the expanded shape can be increased if the deformable portion is cooled to a temperature below room temperature prior to placement in the vasculature of the patient. As the deformable portion or tube 112 reaches 37 degrees Celsius or Tᵣ, the deformable portion or tube 112 transitions or changes shape to the expanded shape, which in the illustrated embodiment (FIG. 4) includes a bulge in deformable portion or tube 112. The shape-change and bulging of deformable portion 112 causes mid-section 133 and distal end 134 (with opening 135) to expand. In the illustrated embodiment, mid-section 133 expands laterally by a greater amount or distance than distal end 134 expands, and distal end 134 expands laterally to a dimension larger than the diameter of body 114. Accordingly, deformable portion or tube 112 resembles a pear in that illustrated expanded configuration. In this form, mid-section 133 is convexly curved, i.e. generally outwardly from catheter 110. Proximal end 132 is either prepared for minimal or no expansion, or its expansion is inhibited by the attachment with body 114 of catheter 110, and so distal end 134 expands a greater amount than proximal end 132. As previously indicated, it will be understood that deformable portion or tube 112 can be configured in other embodiments to expand to form other shapes, such as bell, oval, circular, or other curved shapes.

The expanded shape of deformable portion or tube 112 positioned in the vasculature of a patient has many benefits. Expansion of the deformable portion or tube 112 increases the diameter of the lumen or passageway 140 and the diameter of the opening 135 of distal end 134, to thereby increase the fluid flow rate through deformable portion 112. In particular, as the radius of passageway 140 is doubled, the flow rate through passageway 140 increases fourfold. If aspiration is performed through catheter 110, a larger passageway 140 and distal end 134 of deformable portion 112 help attain laminar flow as compared to catheters having a constant-sized distal end. Expansion of mid-section 133 and distal end 134 ensures that if deformable portion 112 contacts the vessel wall, it would do so with rounded mid-section 133 or the rounded shape of distal end 134. The curvature limits or prevents damage to the vessel wall from any contact of either of these parts with the vessel wall. Further, if aspiration is performed, expanded mid-section 133 and distal end 134 deter distal opening 135 of deformable portion 112 from contacting the vessel wall. In other words, during aspiration, distal end 134 avoids being sucked up against the vessel wall compared to distal ends of traditional straight catheter tips because the expanded shape of deformable portion or tube 112 helps to stabilize the position of the deformable portion or tube 112 in the vasculature, and to maintain opening 135 further from a vessel wall.

Turning now to the assembly or manufacture of catheter assembly 100, deformable portion 112 in the original compact shape can be attached or bonded with catheter 110 by many techniques. In particular embodiments, proximal end 132 of deformable portion 112 is bonded to distal end 118 of catheter 110. Some forms of bonding include using radio-frequency welding, molding, adhesive, or similar techniques that permanently join deformable portion 112 to distal end 118. After proximal end 132 of deformable portion 112 is attached to distal end 118 of catheter 110, heat is applied to deformable portion 112 until it reaches about 37 degrees Celsius or Tᵣ. At this temperature, deformable portion or tube 112 is formed or configured into a desired expanded shape, which as illustrated in FIG. 4 may be a round or bulging configuration roughly in the shape of a pear. As previously mentioned, the expanded shape for deformable portion or tube 112 can include bell, oval, circular, or other shape(s) that enlarge a portion or all of passageway 140 and opening 135 and expands the outer extent one or both of mid-section 133 and distal end 134 at least laterally. During the heating step, deformable portion 112 is thermoset or cured. Once deformable portion 112 is thermoset, it is cooled to a temperature less than 37 degrees Celsius or Tₛ, wherein it returns to or toward the original compact shape (e.g. the cylinder of FIG. 2). After deformable portion 112 returns to the original compact shape, it and the rest of catheter assembly 100 is ready for sterilization and packaging (if needed) and insertion into a patient.

One technique often used to insert a catheter into a vein includes a percutaneous entry technique, such as the Seldinger technique. Catheter assembly 100 described above can also be inserted using this technique or other standard techniques. In the Seldinger technique, the physician makes an oblique entry into the vein with a beveled needle. A wire guide is then inserted through the bore of the needle about 5 to 10 cm into the vein. The needle is thereafter withdrawn, leaving the wire guide in place. An introducer sheath is introduced over the wire guide. Catheter assembly 100 is then introduced into the vein via the introducer sheath and over the wire guide. The wire guide and introducer sheath are removed in a conventional fashion, leaving catheter assembly 100 in the vein. In one embodiment, during insertion into the vein, deformable portion or tube 112 behaves similarly to a conventional catheter tip, so the physician will not have to make adjustments to the required or preferred technique of insertion. After insertion into the vein, the temperature of deformable portion 112 is raised to the patient's body temperature which causes the deformable portion 112 to assume the expanded shape. Warming of deformable portion 112 occurs through transfer of body heat from blood flow and/or adjacent tissues or environment, or can be introduced via another medium or structure. In another embodiment, the deformable portion or tube 112 assumes the expanded shape as the deformable portion or tube 112 travels over the guide wire and introducer sheath. In this embodiment, less trauma to the vasculature is incurred during placement of the catheter assembly 100. In yet another embodiment, a sheath is placed over the deformable portion or tube 112 while the catheter assembly 100 is positioned in the vein to retain the deformable portion or tube 112 in the original compact shape. When it is required or desired to enable the deformable portion or tube 112 to assume an expanded shape, the sheath is removed. When it is required or desired to remove catheter assembly 100 from the patient, deformable portion is cooled to resume or contract toward its original configuration. For example, cool liquid (e.g. plasma or saline from about 20 to about 30 degrees Celsius) may be passed through lumen 124 to deformable portion 112, to thereby lower the temperature of deformable portion 112 below 37 degrees Celsius. When deformable portion or tube 112 is cooled to a temperature below 37 degrees Celsius (in this example), it returns to its original compact shape. Catheter assembly 100 can be removed from the patient by a conventional technique.

Types of shape memory polymers useful in deformable portion 112 include, but are not limited to, polyurethane, polyurethanes with ionic or mesogenic components, block copolymers consisting of polyethylene terephthalate (PET) and polyethylene oxide (PEO), block copolymers containing polystyrene and poly(1,4-butadiene), and an ABA triblock copolymer made from poly(2-methyl-2-oxazoline) and poly(tetrahydrofuran), and chemically crosslinked shape memory polymer materials. Polyurethane material has been found particularly useful.

While the disclosure has been illustrated and described in detail in the drawings and foregoing description, the same is to be considered as illustrative and not restrictive in character, it being understood that only the preferred embodiment has been shown and described and that all changes, equivalents, and modifications that come within the spirit of the disclosures defined by following claims are desired to be protected. All publications, patents, and patent applications cited in this specification are herein incorporated by reference as if each individual publication, patent, or patent application were specifically and individually indicated to be incorporated by reference and set forth in its entirety herein.

1. A catheter assembly, comprising:
a catheter having a distal end opposite a proximal end and a lumen extending between the distal end and the proximal end; and
a deformable portion having a proximal end, a distal end, a mid-section between them and a lumen, said proximal end of said deformable portion attached to the distal end of the catheter so that said lumen of said catheter and said lumen of said deformable portion communicate with each other at a common diameter, said distal end of said deformable portion having an opening to said lumen, the deformable portion having a compact shape at a first temperature and an expanded shape at a second temperature, wherein the second temperature is greater than the first temperature,
and wherein in said expanded shape said mid-section has a diameter larger than said distal end of said deformable portion and said proximal end of said deformable portion, so that when said deformable portion is within a body, said expanded mid-section has a convex external surface that maintains at least part of said opening away from a tissue surface.

2. The assembly of claim 1, wherein the second temperature is about 37 degrees Celsius.

3. The assembly of claim 1, wherein the compact shape of the deformable portion is substantially cylindrical.

4. The assembly of claim 1, wherein the mid-section is configured to expand more than the distal end of the deformable portion.

5. The assembly of claim 4, wherein the distal end of the deformable portion is configured to expand more than the proximal end of the deformable portion.

6. The assembly of claim 1, wherein the deformable portion is made of a shape memory polymer material.

7. The assembly of claim 1, wherein the expanded shape of the deformable portion is a pear shape.

8. A catheter assembly, comprising:
a catheter having a distal end opposite a proximal end and a lumen extending between the distal end and the proximal end; and
a deformable portion having a proximal end, a distal end, a mid-section between them and a lumen, said proximal end of said deformable portion attached to the distal end of the catheter so that said lumen of said catheter and said lumen of said deformable portion communicate with each other at a common diameter, said distal end of said deformable portion having an opening to said lumen, the deformable portion configured to transform from a compact shape to an expanded shape when the deformable portion is heated to a temperature of about 37 degrees Celsius, and wherein in said expanded shape said mid-section has a diameter larger than said distal end of said deformable portion and said proximal end of said deformable portion, so that when said deformable portion is within a body, said expanded mid-section has a convex external surface that maintains at least part of said opening away from a tissue surface.

9. The assembly of claim 8, wherein the deformable portion is made of a shape memory polymer material.

10. The assembly of claim 8, wherein the distal end of the deformable portion is configured to expand more than the proximal end of the deformable portion.

11. The assembly of claim 8, wherein the mid-section of the deformable portion is configured to expand more than the proximal end of the deformable portion.

12. The assembly of claim 11, wherein a part of the deformable portion near the mid-section expands more than the distal end of the deformable portion.

13. The assembly of claim 8, wherein the compact shape of the deformable portion and the distal end of the catheter each have a cross-sectional shape and cross-sectional size, the shapes being about the same and the sizes being about the same.

14. The assembly of claim 8, wherein the deformable portion is configured to transform from the expanded shape to a substantially cylindrical compact shape when the deformable portion is cooled to a temperature less than about 37 degrees Celsius.

15. A method for making a catheter assembly, comprising:
attaching a deformable portion made of a shape memory polymer material to a distal end of a catheter, the deformable portion being in a compact shape;
heating the deformable portion to about 37 degrees Celsius;
transforming the deformable portion from a compact shape to an expanded shape wherein a mid-section of the deformable portion has a diameter larger than a distal end of the deformable portion and a proximal end of the deformable portion, so that the expanded mid-section has a convex external surface between the distal end and the proximal end; and
cooling the deformable portion to less than 37 degrees Celsius wherein the deformable portion returns to the compact shape.

16. The method of claim 15, wherein the attaching the deformable portion includes attaching a proximal end of the deformable portion to the distal end of the catheter.

17. The method of claim 16, wherein the attaching the deformable portion includes gluing the deformable portion with the distal end of the catheter.

18. The method of claim 15, wherein the heating step includes curing the deformable portion.
